(19) United States
(12) Patent Application Publication
(10) Pub. No.: US 2009/0035381 A1
(43) Pub. Date: Feb. 5, 2009

(54) ELECTROSPRAYING METHOD FOR FABRICATION OF PARTICLES AND COATINGS AND TREATMENT METHODS THEREOF

(76) Inventors: John J. Stankus, Campbell, CA (US); Shubhayu Basu, Mountain View, CA (US)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 11/832,257

(22) Filed: Aug. 1, 2007

(51) Int. Cl. A61K 47/30; A61K 38/18; A61K 9/14; B29B 13/08; B29B 9/00

(52) U.S. Cl. 424/501; 264/10; 425/133.1; 514/2; 514/772.3

(57) ABSTRACT

Electrospray systems and modified electrospray systems for the fabrication of core-shell particles for controlled-release and/or sustained-release treatment and delivery are herein disclosed. The electrospray system may include between one and a plurality of co-axially situated tubes. Each tube may be electrically connected to a power supply wherein a voltage may be applied thereto. Core-shell particles may be collected on a collection target, which may be a wet or dry collector, and electrically connected to the power supply. Core-shell particles and methods of manufacture are also disclosed. The precursors of the core-shell particles may be polymer- or biomacromolecule-based solutions and may include at least one treatment agent incorporated therein. The number of “core” particle(s) within the “shell” may vary and may provide different treatment agent release profiles depending on the material and/or chemical characteristics of the polymer and/or biomacromolecule used. Methods of treating a condition are also disclosed. A treatment may include delivery of a plurality of core-shell particles which include a treatment agent to a treatment site. Delivery may be performed by a surgical procedure or by a non-invasive procedure such as catheter delivery.

### FIELD OF INVENTION

Fabrication of core-shell particles for controlled-release and/or sustained-release treatment agent delivery using electrospraying methods.

### BACKGROUND OF INVENTION

Ischemic heart disease typically results from an imbalance between the myocardial blood flow and the metabolic demand of the myocardium. Progressive atherosclerosis with increasing occlusion of coronary arteries leads to a reduction in coronary blood flow, which creates ischemic heart tissue. “Atherosclerosis” is a type of arteriosclerosis in which cells including smooth muscle cells and macrophages, fatty substances, cholesterol, cellular waste product, calcium and fibrin build up in the inner lining of a body vessel. “Arteriosclerosis” refers to the thickening and hardening of arteries. Blood flow may be further decreased by additional events such as changes in circulation that lead to hypoperfusion, vasospasm or thrombosis.

Myocardial infarction (MI) is one form of heart disease that results from the sudden lack of supply of oxygen and other nutrients. The lack of blood supply is a result of a closure of the coronary artery (or any other artery feeding the heart) which nourishes a particular part of the heart muscle. The cause of this event is generally attributed to arteriosclerosis in coronary vessels.

Formerly, it was believed that an MI was caused from a slow progression of closure from, for example, 95% then to 100%. However, an MI may also be a result of minor blockages where, for example, there is a rupture of the cholesterol plaque resulting in blood clotting within the artery. Thus, the flow of blood is blocked and downstream cellular damage occurs. This damage may cause irregular rhythms that may be fatal, even though the remaining muscle is strong enough to pump a sufficient amount of blood. As a result of this insult to the heart tissue, scar tissue tends to naturally form. Additionally, the myocardium wall may become thinned and dilated, ultimately leading to heart failure.

An important component in the progression to heart failure is remodeling of the heart due to mismatched mechanical forces between the infarcted region and the healthy tissue resulting in uneven stress and strain distribution in the left ventricle. Once an MI occurs, remodeling of the heart begins. The principle components of the remodeling event include myocyte death, edema and inflammation, followed by fibroblast infiltration and collagen deposition, and finally scar formation from extra-cellular matrix (ECM) deposition. The principle component of the scar is collagen which is non-contractile and causes strain on the heart with each beat. Non-contractility causes poor pump performance as seen by low ejection fraction (EF) and akinetic or diskinetic local wall motion. Low EF leads to high residual blood volume in the ventricle, causes additional wall stress, and leads to eventual infarct expansion via scar stretching and thinning and border-zone cell apoptosis. In addition, the remote-zone thickens as a result of higher stress which impairs systolic pumping while the infarct region experiences significant thinning because mature myocytes of an adult are not regenerated. Myocyte loss is a major etiologic factor of wall thinning and chamber dilation that may ultimately lead to progression of cardiac myopathy. In other areas, remote regions experience hypertrophy (thickening) resulting in an overall enlargement of the left ventricle. This is the end result of the remodeling cascade. These changes also correlate with physiological changes that result in increase in blood pressure and worsening systolic and diastolic performance.

Previous and current methods to treat MI have been aimed at developing methods to sustain or repair damaged myocardium. These methods include the delivery of appropriate cells to the injury site, delivery of biomaterials, or delivery of cell-loaded biomaterials. Limitations of current technologies include low cell retention at the injection side and reduced long term viability. By adding cells into a biomaterial before application it is believed that retention may be improved. These biomaterials may include but are not limited to fibrin glue, alginate gel, and synthetic polyethylene glycol based materials. Further, by supplementing these cells with appropriate drugs or growth factors, they may be stimulated to better survive in vivo. Previous methods under consideration for drug loading include liposomal and micro- or nanoparticle delivery carriers.

Microparticulate controlled release systems fabricated from degradable polymers have been widely investigated for the delivery of drugs over recent years. Some research has been directed at substituting a drug with a protein or peptide to exploit such systems. Challenges associated with such system include enzymatic degradation in oral delivery and permeating across gastrointestinal epithelial layers in localized delivery.

One method for fabricating microparticles exploits a (water-in-oil) in-water (W/O/W) double emulsion method. In the WI phase, an aqueous phase containing treatment agent (i.e., a protein or peptide), is dispersed into the oil phase consisting of polymer dissolved in organic solvent (e.g., dichloromethane) using a high-speed homogenizer. Examples of sustained-release polymers include poly(D,L-lactide-co-glycolide) (PLGA), poly(D,L-lactide) (PLA), poly(ε-caprolactone) or PLA-PEEK co-polymers, poly(ester-amide) (PEA). The primary water-in-oil (W/O) emulsion is then dispersed within an aqueous solution containing a polymeric surfactant, e.g., poly(vinyl alcohol) (PVA), and further homogenized to produce a W/O/W emulsion. After stirring for several hours, the microparticles are collected by filtration.

The exposure of a protein to various factors unfavorable for stability, such as organic solvents and polymer degradation, may promote deactivation during the W/O/W process. For example, the microencapsulation of the enzyme carbonic anhydrase within PLGA microspheres has been reported to cause severe non-covalent aggregation upon exposure to the water/oil interface, while the encapsulated enzyme was severely hydrolysed within fast degrading PLGA due to acidic microenvironment generated from polymer degradation.

Single and double emulsions may also be suitable to deliver substances in a controlled manner. Among methods to generate emulsions, processes involving bulk processes require long time multi-step procedures which generally result in emulsions with wide droplet size distributions. In recent alternative approaches, the liquid interface is smoothly stretched out by the action of either capillary, hydrodynamic, or electrical forces until its characteristic length reaches a critical value (usually in the micro or nanometric range) and the interface breaks by capillary instabilities yielding substantially monodisperse drops. Such approaches include: (i) dripping or jetting throughout micro-orifices, (ii) hydrodynamic focusing, i.e., the stretching out of an interface by the high velocity gradients of a converging flow, and (ii) the use of electrohydrodynamic forces for the stretching out of the interface. The two first methods permit sufficient control of the size of the dispersed phase that may even be extended to the droplet shape by the use of copolymers and induced polymerization. In methods of type (i), the distribution of the droplet size is quite narrow; nonetheless, the characteristic cross-section of the capillaries (or channels), and therefore, the size of the obtained drops, is limited to a few microns to avoid clogging problems. On the other hand, devices based on hydrodynamic focusing (ii) are free of the above limitation, although the narrowest size distribution is obtained when the mean size of the droplets is about few tens of microns. Both methods (i) and (ii) have been also employed to obtain double emulsions, with sufficient control on both the size of the dispersed phase and the structure of the droplets.

### SUMMARY OF INVENTION

Electrospray systems and modified electrospray systems for the fabrication of core-shell particles for controlled-release and/or sustained-release treatment and delivery are herein disclosed. The electrospray system may include between one and a plurality of co-axially situated tubes within a larger tube. Each co-axially situated tube may independently be in fluid connection with a pumping station. In one embodiment, a first tube is co-axially situated within a second tube which is co-axially situated within a third tube. In another embodiment, a plurality of tubes are co-axially situated within a larger tube in a multitude of arrays. Each tube may be electrically connected to a power supply wherein a voltage may be applied thereto. Core-shell particles may be collected on a collection target, which may be a wet or dry collector, and electrically connected to the power supply.

Core-shell particles and methods of manufacture are also disclosed. The precursors of the core-shell particles may be polymer- or biomacromolecule-based solutions and may include at least one treatment agent incorporated therein. The number of “core” particle(s) within the “shell” may vary and may provide different treatment agent release profiles depending on the material and/or chemical characteristics of the polymer and/or biomacromolecule used.

Methods of treating a condition are also disclosed. A treatment may include delivery of a plurality of core-shell particles which include a treatment agent to a treatment site. Delivery may be performed by a surgical procedure, such as open chest surgery, or by a non-invasive procedure such as catheter delivery.

### BRIEF DESCRIPTION OF DRAWINGS

FIG. 1 illustrates a plan view of an electrospray system.

FIG. 2 illustrates a plan view of an alternative electrospray system.

FIG. 3A illustrates a plan view of an embodiment of an extrusion-modified electrospray system in accordance with embodiments of the invention.

FIG. 3B illustrates a top view of the extrusion-modified electrospray system of FIG. 3A.

FIGS. 4A-4B illustrate an alternative embodiment of an extrusion-modified electrospray system in accordance with embodiments of the invention.

FIG. 5 illustrates an alternative embodiment of an extrusion-modified electrospray system in accordance with embodiments of the invention.

FIGS. 6A-6B illustrate a cross-sectional view of an embodiment of multi-array nozzle.

FIG. 6C illustrates an embodiment of a core-shell particle in accordance with embodiments of the invention.

FIGS. 7A-7B illustrate a cross-sectional view of an embodiment of multi-array nozzle.

FIG. 7C illustrates an alternative embodiment of a core-shell particle in accordance with embodiments of the invention.

FIGS. 8A-8C illustrate an alternative embodiment of a dual-needle injection device which may be used to deliver core-shell particles in accordance with embodiments of the invention.

FIG. 9 illustrates an embodiment of a syringe which may be used pursuant to embodiments of the invention.

### DETAILED DESCRIPTION

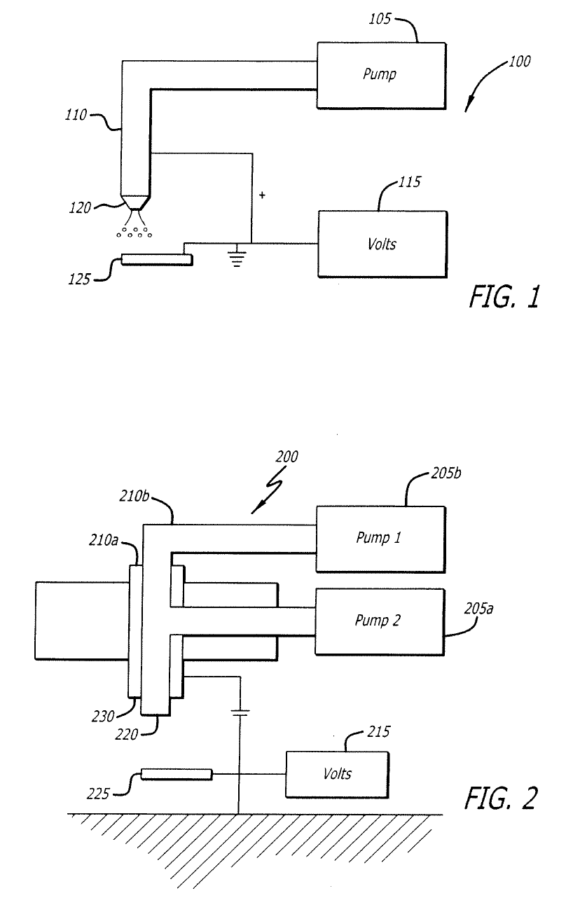

Electrospraying is a method based on the ability of an electric field to overcome the surface tension of a polymer or biomacromolecule solution (or melt). Electrospray uses electricity to form charged droplets which are generally collected on a collection plate. FIG. 1 illustrates generally electrospray system 100. Electrospray system 100 includes pump 105 connected to hollow capillary tube 110. High voltage power supply 115 is connected to hollow capillary tube 110 which is generally made of a conductive metal. Power supply 115 supplies a charge to hollow capillary tube 110 which in turn charges the liquid solution passing therethrough. As a solution is pumped through hollow capillary tube 110 and exits nozzle 120, the solution is collected as particles on collection target 125, which is generally grounded.

In conventional electrospraying methods, a liquid is injected through hollow capillary tube 110 in an external medium (gas, vacuum, or a dielectric liquid). Depending on various process parameters, such as flow rate and the electric voltage applied between the needle and a grounded electrode, the liquid meniscus at the end of the needle adopts a conical shape resulting from the balance between the capillary and the electrohydrodynamic normal stresses. This conical shape is referred to as a Taylor cone. Eventually, a micro- or nanometric jet issues from the tip of the Taylor cone, which will eventually break up forming a spray (or hydrosol) of charged droplets. The droplets are collected on collection target 125 as particles.

In some embodiments, an electrospray system may include coaxially situated hollow capillary tubes, or cylindrical members (hereinafter used interchangeably). FIG. 2 illustrates electrospray system 200 including outer tube 210a, i.e., cylindrical member, terminating in annular opening 230 and inner tube 210b terminating in tip or nozzle 220 (hereinafter used interchangeably). In one embodiment, outer tube 210a may be insulating and inner tube 210b may be conductive. Examples of materials that may comprise insulating outer tube 210a include polyether ether ketone (PEEK), polytetrafluoroethylene (PTFE), fluorinated ethylene propylene, high density polyethylene (HDPE), polypropylene, and glass. Materials that may comprise conductive inner tube 210b include stainless steel, aluminum or copper, or Nitinol. In other embodiments, both outer tube 210a and inner tube 210b may be both conductive or, alternatively, both insulating. As with conventional electrospray systems, a charge may be applied to inner tube 210b supplied by power supply 215 while collection target 225 is grounded. Alternatively, an opposite charge to that of inner tube 210b may be applied to collection target 225. In another embodiment, the solution which passes through inner tube 210b may be subjected to a charge or may inherently carry a charge, e.g., a conductive polymer. In either case, a positive charge is generally applied to the solution or to outer tube 210a.

In some embodiments, a first liquid solution (L₁) may be supplied to outer tube 210a by pump 205a and a second different liquid solution (L₂) may be supplied to inner tube 210b by pump 205b to form core-shell particles. Solution L₁ may be the precursor solution that forms the “shell” while solution L₂ may be the precursor solution that forms the “core” of the particles that will be eventually collected on collection target 225 as electrospray system 200 is being operated. By creating core-shell particles encapsulating a therapeutic agent, different release profiles may be obtained as the core and shell independently (or not independently) erode after delivery to a treatment site over a period of time (condition dependent).

Both core and shell materials may include a material that is thermoplastic, biocompatible and bioerodable. “Thermoplastic” is a property wherein the material is soft and pliable when heated. “Biocompatible” means that the material has the capability of co-existing with living tissues or organisms without causing substantial harm. “Bioerodable” means that the material has the capability to degrade over time under physiological conditions. Examples of such materials include, but are not limited to, polymers and biomacromolecules.

In some embodiments, the shell material may be a hydrophobic material, while the core material may be a hydrophilic material. Examples of hydrophobic and hydrophilic polymers include, but are not limited to, polypropylene; polypropyleneglycol (PPG); polyvinylpyrrolidone (PVP); poly(ester amide) (PEA); acrylic acid (AA); polyacrylates such as poly(methyl methacrylate) (PMMA), poly(butyl methacrylate), poly(ethyl methacrylate), hydroxyethylmethacrylate (HEMA), poly(ethyl methacrylate-co-butyl methacrylate) (P(MMA-co BMA)), ethyl glycol dimethacrylate, (EGDMA) and ethylene-methyl methacrylate copolymers; acrylamides such as N,N-dimethyl acrylamide, diacetone acrylamide, and acrylamide-methyl-propane sulfonate (AMPS); fluorinated polymers or copolymers such as poly(vinylidene fluoride) and poly(vinylidene fluoride-co-hexafluoro propene); poly(N-vinyl pyrrolidone); poly(N-vinyl pyrrolidone-co-vinyl acetate); poly(hydroxyvalerate); poly(L-lactic acid)/polylactide (PLLA); poly(ε-caprolactone); poly(L-lactide-co-caprolactone); poly(lactide-co-glycolide) (PLGA); poly(hydroxybutyrate); poly(hydroxyvalerate); poly(hydroxybutyrate-co-valerate); polydioxanone; polyorthoester; polyanhydride; poly(glycolic acid)/polyglycolide (PGA); poly(D,L-lactic acid) (PLA); poly(glycolic acid-co-trimethylene carbonate); polyphosphoester; polyurethanes such as polyphosphoester urethane, poly(amino acids); cyanoacrylates; poly(trimethylene carbonate); poly(iminocarbonate); co-poly(ether-esters); polyalkylene oxalates; polyphosphazenes; silicones; polyesters; polyolefins; polyisobutylene and ethylene-alphaolefin copolymers; vinyl halide polymers and copolymers such as polyvinyl chloride (PVC); polyvinyl ethers such as polyvinyl methyl ether; polyvinylidene chloride; polyacrylonitrile; polyvinyl ketones; polyvinyl aromatics such as polystyrene, styrene sulfonate and acrylonitrile-styrene copolymers; polyvinyl esters such as polyvinyl acetate; copolymers of vinyl monomers with each other such as divinyl benzene (PVB); olefins such as poly(ethylene-co-vinyl alcohol) (EVAL); acrylonitrile butadiene (ABS) resins; and ethylene-vinyl acetate copolymers; polyamides such as Nylon 66 and polycaprolactam; alkyd resins; polycarbonates; polyoxymethylenes; polyimides; polyethers; epoxy resins; polyurethanes polyurethane(ureas); biodegradable polyurethanes; biodegradable polyurethane(ureas); rayon; and rayon-triacetate, poly(ethylene glycol) (PEG), and poly(vinyl alcohol) (PVA).

A biomacromolecule may include, but is not limited to, fibrin; fibrinogen; dextran; cellulose including cellulose, cellulose acetate, cellulose butyrate, cellulose acetate butyrate, cellophane, cellulose nitrate, cellulose propionate, cellulose ethers, and carboxymethyl cellulose; starch; pectin; chitosan; gelatin; alginate and conjugations thereof including alginate-gelatin, alginate-collagen, alginate-laminin, alginate-elastin, alginate-collagen-laminin and alginate-hyaluronic acid; collagen and conjugates thereof; hyaluronan; hyaluronic acid; sodium hyaluronate; modified hyaluronan such as tyramine-hyaluronate or glycidyl methacrylate hyaluronate; or self-assembled peptides (SAP) such as AcN-RARADADARARADADA-CNH₂ (RAD 16-II), VKVKVKVKV-PP-TKVKVKVKV-NH₂ (MAX-1), and AcN-AEAEAKAKAEAEAKAK-CNH₂ (EAK 16-II).

In some embodiments, a treatment agent or a biologic (hereinafter used interchangeably) may be incorporated within solution L₁ or solution L₂, or both solutions L₁ and L₂. For example, the treatment agent may be physically dissolved or covalently attached to the polymers or biomacromolecules which are solvated within the precursor solutions prior to fabrication of the particles. Examples of treatment agents and/or biologics may include, but are not limited to, an anti-proliferative, an anti-inflammatory or immune modulating agent, an anti-migratory, an anti-thrombotic or other pro-healing agent, or a combination thereof. More specific examples of treatment agents typically used to treat heart failure may include small molecule drugs such as, for example, angiotensin or an angiotensin-converting enzyme (ACE) inhibitors, angiotensin receptor blockers (ARBs), beta-blockers, diuretics, digoxin, hydralzine, and dobutamine.

The anti-proliferative agent may be a natural proteineous agent such as cytotoxin or a synthetic molecule or other substances such as actinomycin D, or derivatives and analogs thereof (manufactured by Sigma-Aldrich 1001 West Saint Paul Avenue, Milwaukee, Wis. 53233; or COSMEGEN available from Merck) (synonyms of actinomycin C1); all taxoids such as taxols, docetaxel, and paclitaxel, and paclitaxel derivatives; all olimus drugs including macrolide antibiotics such as tacrolimus, rapamycin (i.e., sirolimus) derivatives of which include 40-O-[2-(2-hydroxy)ethoxy]ethyl-rapamycin, 40-O-(3-hydroxy)propyl-rapamycin, 40-O-tetrazole-rapamycin, 40-epi-(N1-tetrazolyl)-rapamycin (ABT-578 manufactured by Abbott Laboratories, Abbott Park, Ill.); everolimus (i.e., RAD-001); FKBP-12 mediated mTOR inhibitors, perfenidone and prodrugs, co-drugs and combinations thereof. An anti-proliferative may be used in, for example, a cancer treatment.

The anti-inflammatory agent may be a steroidal anti-inflammatory agent, a nonsteroidal anti-inflammatory agent, or a combination thereof. In some embodiments, anti-inflammatory drugs include, but are not limited to, alclofenac, alclometasone diproprionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen fenclofenac, fenclorac, fendosal, fenpipalone, fentiazac, flazalone, fluazocort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone sodium glycerate, perfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicyclic acid), salicyclic acid, corticosteroids, glucocorticoids, tacrolimus, pimecorlimus, prodrugs thereof, co-drugs thereof, and combinations thereof.

These agents may also have anti-proliferative and/or anti-inflammatory properties or may have other properties such as antineoplastic, antiplatelet, anti-coagulant, anti-fibrin, antithrombonic, antimitotic, antibiotic, antiallergic, antioxidant and/or cytostatic (i.e. cell-suppressing) properties. Examples of suitable treatment and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Other examples of biologics include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Examples of antineoplastics and/or antimitotics include methotrexate, azathioprine, vincristine, vinblastine, fluorouracil, doxorubicin hydrochloride (e.g., Adriamycin® from Pharmacia & Upjohn, Peapack, N.J.), and mitomycin (e.g., Mutamycin® from Bristol Myers Squibb Co, Stamford, Conn.). Examples of such antiplatelets, anticoagulants, antifebrin, antithrombins include sodium heparin, low molecular weight heparins, heparinoids, hirudin, argatroban, forskolin, vapiprost, prostacyclin, and prostacyclin analogues, dextran, D-phe-pro-arg-chloromethylketone (synthetic antithrombin), dipyridamole, glycoprotein 11b/111a platelet membrane receptor antagonist antibody, recombinant hirudin, thrombin inhibitors such as Angiomax a (Biogen, Inc. Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacort from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, dietary supplements such as various vitamins, and a combination thereof. Examples of cytostatic substances include angiopeptin, angiotensin converting enzyme inhibitors such as captopril (e.g., Capoten® and Capozide® from Bristol Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium. Other therapeutic substances or agents which may be appropriate include α-interferon, and genetically engineered epithelial cells. The foregoing substances are listed by way of example and are not meant to be limiting. Other treatment agents which are currently available or that may be developed in the future are equally applicable.

In some embodiments, the biologic is a protein or combination of multiple proteins such as, but not limited to, vascular endothelial growth factor (VEGF), basic fibroblast growth factor (bFGF), acidic fibroblast growth factor (aFGF), platelet-derived growth factor (PDGF), platelet-derived endothelial growth factor (PDEGF), placental derived growth factor, angiopoietin-1 (Ang-1), angiopoietin-2 (Ang-2), insulin-like growth factor 1 (IGF-1), insulin-like growth factor-2 (IGF-2), muscle derived insulin-like growth factor (mIGF), transforming growth factor-alpha (TGF-α), transforming growth factor-beta (TGF-β), hepatocyte growth factor (HGF), stem cell factor (SCF), hematopoietic growth factor or granulocyte colony-stimulating factors (G-CSF), granulocyte macrophage colony-stimulating factors (GM-CSF), nerve growth factor (NGF), growth differentiation factor-9 (GDF9), epidermal growth factor (EGF), stromal derived growth factor-1α (SDF-1α) neurotrophins, erythropoietin (EPO), thrombopoieten (TPO), myostatin (GDF-8), leukemia inhibitory factor (LIF), tumor necrosis factor-alpha (TNF-α), sonic hedgehog protein (Shh).

In one embodiment, solution L₁ may incorporate a hydrophobic polymer such as poly(lactide-co-glycolide) or poly(ε-caprolactone). Solution L₁ will form the “shell” of the core-shell particle. The hydrophobic polymer may be in a molecular weight range of between about 200 Daltons to about 500,000 Daltons. The concentration may be between about 0.01 mg/mL to about 1000 mg/mL (weight percent) depending on the molecular weight of polymer and solvent utilized. Generally, a higher concentration leads to larger-sized particles. The polymer may be dissolved in an appropriate organic solvent including, but not limited to, acetone, dichloromethane, ethyl acetate, chloroform, tetrahydrofuran, dimethyl sulfoxide, trichloroethane, and hexafluoroisopropanol.

Solution L₂ may incorporate a hydrophilic polymer such as PEG or PVA. Solution L₂ will form the “core” of the core-shell particle. The hydrophilic polymer may be in a molecular weight range of between about 200 Daltons to about 1,500,000 Daltons and the concentration may be between about 0.01 mg/mL to about 1000 mg/mL. The polymer may be dissolved in an appropriated aqueous solvent including, but not limited to, phosphate buffer, Dulbecco's phosphate buffer, HEPES buffer, TRIS buffer, and acetic acid. The viscosity of the hydrophobic polymer solution L₂ will be dependent upon the specific material and the solvent in which the material is dissolved. Furthermore, depending on the platform used (i.e., polymer, biomacromolecule, etc.), solution L₂ may have a certain associated conductivity. For example, solution L₂ may include a conductive polymer. A “conductive polymer” is an organic polymer semiconductor and includes, but is not limited to, polyacetylene, polypyrrole, polyaniline, and their derivatives. Additionally, conductivity of solution L₂ may be increased by the addition of a salt such as sodium chloride, potassium chloride, calcium chloride, magnesium chloride, lithium chloride, sodium carbonate or sodium phosphate. Generally, a more conductive solution will give smaller-sized particles when other electrospray process variables are held constant.

In some embodiments, a treatment agent such as those previously described may be incorporated within solution L₂ or L₂. The amount of treatment agent or biologic should be an amount effective to treat a particular treatment site. For example, VEGF may be added to solution L₂ in a concentration of between about 0.010 μg/mL to about 10,000 μg/mL to treat necrosed tissue at a post-MI treatment region. In some embodiments, an excipient may be added to solution L₁, solution L₂, or both solutions L₁ and L₂, to improve release of the treatment agent or to change the morphology of the particles. Examples of excipients include, but are not limited to, bovine serum albumin (BSA), human serum albumin, trehalose, Pluronic surfactants, PEG and PVA. “Pluronics” are a family of poly(ethylene oxide)-poly(propylene oxide)-poly(ethylene oxide) triblock copolymers (PEO-PPO-PEO). Pluronics are known for their unique surfactant abilities, low toxicity, and minimal immune response. The concentration of the excipients may be from about 0.01 mg/mL to about 500 mg/mL, depending on the release profile desired to be achieved. The different possible morphologies of the particles will be explained in more detail below.

In order to form a completely or substantially monodisperse population of core-shell particles, various process conditions in the operation of, for example, electrospray system 200 as illustrated in FIG. 2, may be controlled and adjusted. For example, the distance from the tip of both annular opening 230 and nozzle 220 to collection target 225 may affect the wetness or dryness of the core-shell particles. For example, a longer distance may result in drier particles collected as the emulsion droplets emerging from the tubes hit collection target 225. As the emulsion droplets travel from the terminal ends of tubes 210a and 210b (i.e., annular opening 230 and nozzle 220), solvent rapidly evaporates from the particle material. In some embodiments, the distance from the terminal ends of tubes 210a and 210b may vary from about 0.01 millimeters (mm) to 2.5 centimeters (cm).

Additionally, the inner diameter (ID) of inner tube 210b may affect the particle size. That is, the smaller the ID, the smaller the particles and vice-versa. In some embodiments, an electric charge may be applied to inner tube 210b. The voltage applied to inner tube 210b may be in a range from between about 0.5 kilovolt (kV) to about 40 kV. Generally, once operating in the critical electrospraying range, higher voltage magnitudes may result in smaller particles until out of the critical range and multi-jetting begins. Additionally, the charge and type of collection target 225 may affect yield. For example, in some embodiments, charging collection target 225 with opposite polarity relative to inner tube 210b allows for increased yields. The voltage applied to collection target 225 may be in a range from between about 0.5 kV to about 40 kV. In some embodiments, a ring electrode or third ring electrode with a polarity that is the same as the spraying solution may allow for better control of spraying by focusing the spray stream for increased particle yield. The ring/third electrode may be placed either near nozzle 220 or collection target 225 or somewhere in between. If ring/third electrode is near nozzle 220, it will possess the same polarity as nozzle 220 albeit a lower magnitude (lower kV) to focus the particle spray and improve size mono-dispersity and particle yield. Alternatively, a ring/third electrode may be a conductive tube surrounding nozzle 220 which is charged with the same polarity and lower magnitude. If placed around or near collection target 225, it would be charged same polarity as collection target 225 to help focus or attract particle spray to collection target 225. For example, a third electrode may be a conductive ring placed inside a glass beaker.

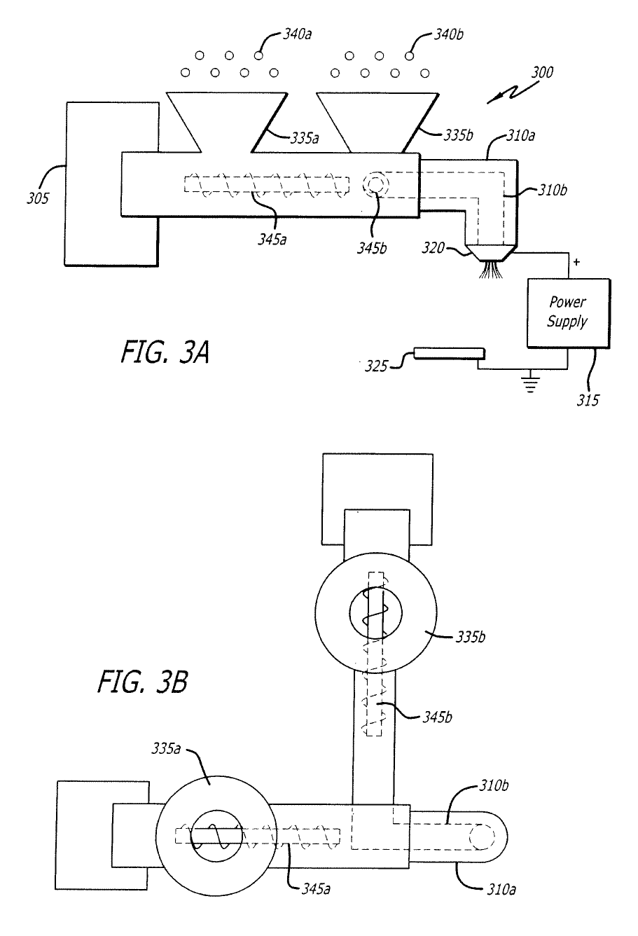

In addition to the distance, the flowrate and temperature of solutions L₁ and L₂ may be adjusted to control particle size, morphology and/or yield. For example, the flow rate of solutions L₁ and L₂ may range from about 0.01 milliliters per hour (mL/h) to about 50 mL/h from a single nozzle. A higher flowrate generally results in larger particles. Temperature may range from about 2-8° C. to about 250° C. Generally, a higher temperature will result in higher solvent evaporation and faster processing and may in some cases be utilized for melt electrospray. It should be appreciated that the process conditions described are equally applicable to alternative embodiments of electrospray systems as discussed below with reference to FIGS. 3-5.

In some embodiments, dry collection of the emerging particles from an electrospray system may be employed. Collection target 225 may be comprised of a material that is conductive metal, a non-conductive material with a conductive metal surface, a conductive metal with a non-conductive surface, or an enclosed chamber with circulating air, such as a cyclone (available from BUCHI Labortechnik AG, Switzerland). A gas such as nitrogen, argon, or air that may also be heated and may be applied to collection target 225 to increase solvent evaporation and/or to increase particle yield with dry filter collection. The flowrate of the gas may be from about 0.5 liters per minute (LPM) to about 200 LPM. A heating lamp or other source may also heat the enclosed chamber in the absence of circulating gas.

In other embodiments, wet collection of the emerging particles from an electrospray system may be employed. In this embodiment, collection target 225 is immersed in a wet bath, such as a beaker. The liquid in the wet bath may be an aqueous solution which may optionally include a surfactant or, alternatively, an organic solvent. Examples of suitable surfactants include, but are not limited to, sodium dodecyl sulfate (SDS), tween20, tween80, Pluronic surfactants, PVA, ammonium lauryl sulfate, benzalkonium chloride and other co-polymers of PEO and PPO. In an aqueous solution, the concentration of surfactant may be between about 0.01% to about 5%. Generally, increasing the surfactant volume will decrease the particle to solvent ratio. Examples of suitable organic solvents include, but are not limited to, ethanol and hexane. In some embodiments, a spin bar may be added to the wet solution. Depending on the spin bar type, size and spinning speed, the morphology of the collected particles may be affected.

One method for harvesting particles wherein wet collection is employed may be as follows: centrifuge at 15000 rpm for 20 minutes; (b) wash with DI water; (c) centrifuge at 15000 rpm for 10 minutes; (d) wash with DI water; (e) centrifuge at 15000 rpm for 10 minutes; (f) wash with DI water; (g) freeze in liquid nitrogen; (h) lyophilize for 48 hours. The centrifuge rpm speed may be lowered if larger particles are being harvested. It should be appreciated that the collection methods (wet and dry) described are equally applicable to alternative embodiments of electrospray systems as discussed below with reference to FIGS. 3-5.

In some embodiments, rather than solutions, pre-fabricated solid polymer materials may be used as the starting materials to form core-shell particles. For example, polymer pellets or a pre-extruded polymer fiber (between about 1.0 mm and 3.0 mm) which have been pre-fabricated with the polymers, biomacromolecules and treatment agents as described previously may be used as the starting materials. Use of such materials may require an extrusion-modified electrospray system.

FIG. 3A illustrates an embodiment of an extrusion-modified electrospray system 300. System 300 includes hopper 335a in which polymer pellets 340a may be fed into for drying thereof. Polymer pellets 340a are heated to a molten state as it is fed into the extrusion tooling, which tooling includes screw driver extruder 345a and screw drive motor 305a. In one embodiment, polymer pellets 340a are hydrophobic material, forming the basis for the “shell” material of extruded core-shell particles. Positioned approximately perpendicular to hopper 335a and shown in FIG. 3B is hopper 335b in which polymer pellets 340b may be fed into for drying thereof. In one embodiment, polymer pellets 340b are hydrophilic material, forming the basis for the “core” material of extruded core-shell particles. The molten polymers flows through co-axial feedpipe 310 and exits out nozzle or “die” 320. Die 320 is charged at high voltage by power supply 315 and discharges particles onto collection target 325, which is generally grounded.

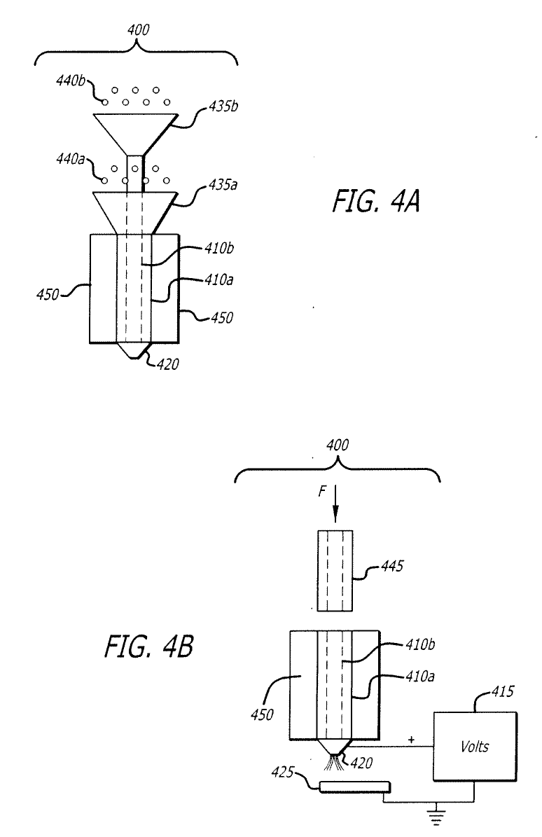

FIGS. 4A-4B illustrate an alternative embodiment of an extrusion-modified electrospray system 400. As shown in FIG. 4A, system 400 includes hopper assembly 435 which includes hopper 435a and hopper 435b in which polymer pellets 440a and polymer pellets 440b, respectively, may be fed into for drying thereof. In one embodiment, polymer pellets 440a are hydrophobic material, forming the basis for the “shell” material of extruded core-shell particles and polymer pellets 440b are hydrophilic material, forming the basis for the “core” material of extruded core-shell particles. Thereafter, hopper 435 is removed and replaced with co-axial piston extruder 445 (FIG. 4B). Polymer pellets 440a and 440b are heated to a molten state in co-axial feedpipe 410 by heating sleeve 450 and charged at high voltage by power supply 415. Piston extruder 445 asserts constant force F on the molten polymer and forces it to exit out die 420, which is also charged at high voltage by power supply 415. Die 420 discharges particles onto collection target 425, which is generally grounded.

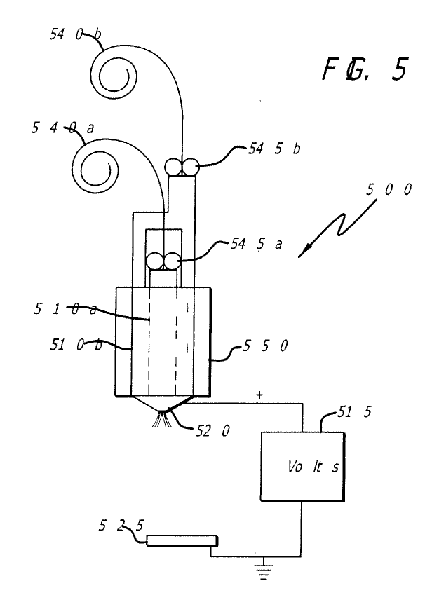

FIG. 5 illustrates another embodiment of an extrusion modified electrospray-system 500. System 500 includes roller mechanisms 545a and 545b in which pre-extruded polymer fibers 540a and 540b may be fed into, respectively, for drying thereof.

In one embodiment, polymer fiber 540a is hydrophilic material, forming the basis for the “core” material of extruded core-shell particles and polymer fiber 540b is hydrophobic material, forming the basis for the “shell” material of extruded core-shell particles. Polymer fibers 540a and 540b are heated to a molten state as it is fed into coaxial feedpipe 510 which is heated by heating sleeve 550. The molten polymers flow through co-axial feedpipe 510 and exits out die 520. Die 520 is charged at high voltage by power supply 515 and discharges particles onto collection target 525, which is generally grounded.

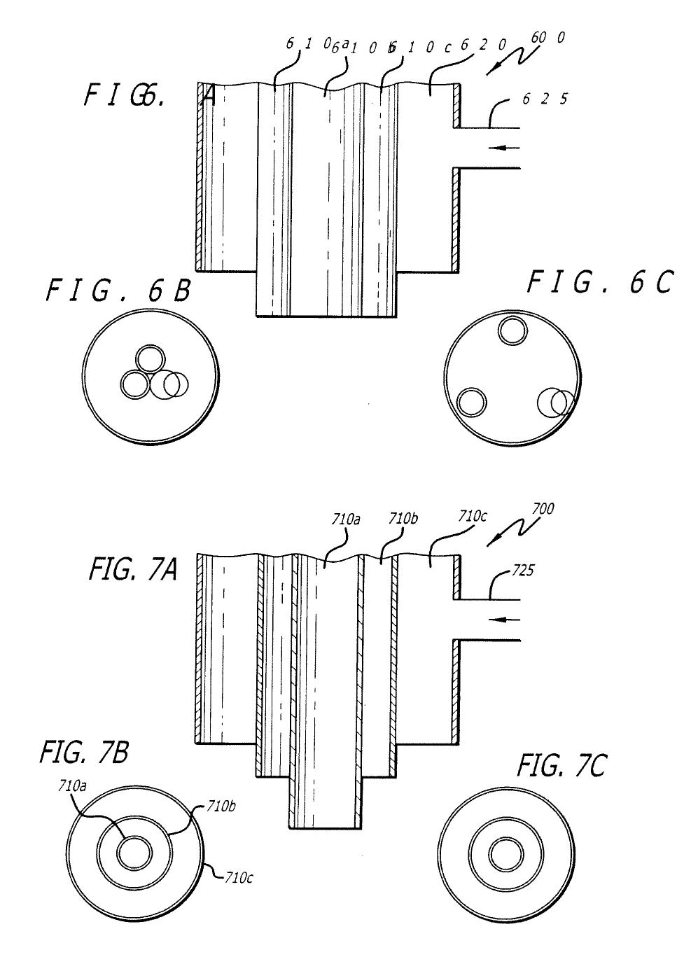

In some embodiments, the nozzle described above in various embodiments may be modified to create different geometries of the resulting collected particles. FIG. 6A illustrates a cross-sectional view of an embodiment of multi-array nozzle 600 with at least three inner hollow capillary tubes 610a, 610b and 610c. Tubes 610a, 610b and 610c may provide channels for various liquid solutions L₃, L₄ and L₅ for fluid passage therethrough, which may be the precursor solutions that will eventually form various “core” particles. Similarly, outer hollow capillary tube 620 provides a channel for a liquid solution L₆ which may be the precursor solution(s) of the “shell” of core-shell particles with multiple cores. The solutions may be those described previously. In one embodiment, solutions L₃, L₄ and L₅ may be comprised of a hydrophilic polymer with at least one treatment agent incorporated therein, while solution L₆ may be comprised of a hydrophobic polymer with or without a treatment agent. Feedtube 625 is fluidly connected with tube 620 for passage of solution L₆ from feedtube 625 to tube 620. Feedtubes fluidly connected to tubes 610a, 610b and 610c are not shown in this illustration. FIG. 6B illustrates a bottom view of multi-array nozzle 600. FIG. 6C illustrates an anticipated multi-core core-shell particle which may result from nozzle 600 under certain process conditions.

FIG. 7A illustrates a cross-sectional view of an alternative embodiment of multi-array nozzle 700. Nozzle 700 may include at least three capillary tubes 710a, 710b and 710c which may be situated co-axial relative to one another. Tubes 710a and 710b may provide channels for various liquid solutions L₇ and L₈ for fluid passage therethrough, and may each form the basis of various “core” particles. Similarly, outer hollow capillary tube 710c provides a channel for a liquid solution L₉ which may form the basis of the “shell” of core-shell particles with multiple cores. The solutions may be those described previously. In one embodiment, solutions L₇ and L₈ may be comprised of a hydrophilic polymer with at least one treatment agent incorporated therein, while solution L₉ may be comprised of a hydrophobic polymer with or without a treatment agent. Feedtube 725 is fluidly connected with tube 710c for passage of solution L₉ from feedtube 725 to tube 710c. Feedtubes fluidly connected to tubes 710a and 710b are not shown in this illustration. FIG. 7B illustrates a bottom view of multi-array nozzle 700. FIG. 7C illustrates an anticipated multi-core core-shell particle which may result from nozzle 700 under certain process conditions.

### Methods of Treatment

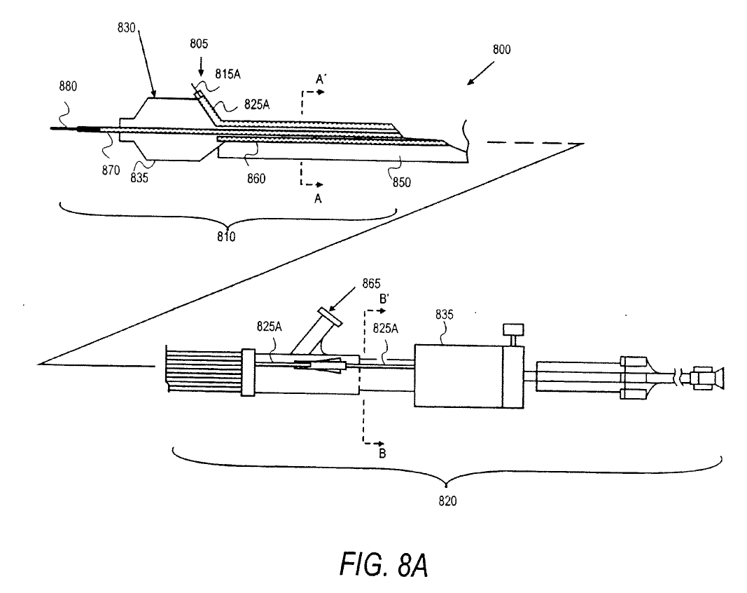

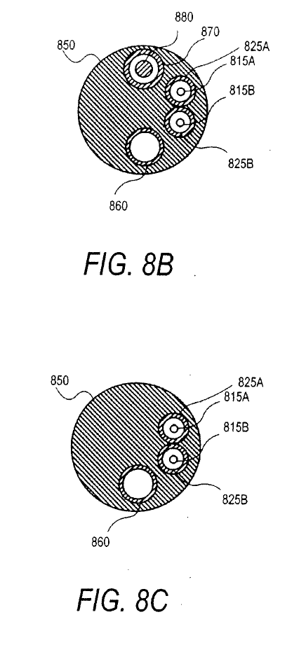

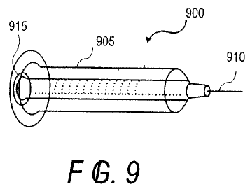

FIGS. 8A-8C illustrate an alternative embodiment of a dual-needle injection device which may be used to deliver core-shell particles in accordance with embodiments of the invention. In general, the catheter assembly 800 provides a system for delivering substances, such as core-shell particles in a delivery vehicle (i.e., gel, solution), to or through a desired area of a blood vessel, in particular, a coronary artery, or organ in order to treat a myocardial infarct region (or other treatment region). The catheter assembly 800 is similar to the catheter assembly described in commonly-owned, U.S. Pat. No. 6,554,801, titled “Directional Needle Injection Drug Delivery Device,” which is incorporated herein by reference.

In one embodiment, catheter assembly 800 is defined by elongated catheter body 850 having proximal portion 820 and distal portion 810. Guidewire cannula 870 is formed within catheter body (from proximal portion 810 to distal portion 820) for allowing catheter assembly 800 to be fed and maneuvered over guidewire 880. Balloon 830 is incorporated at distal portion 810 of catheter assembly 800 and is in fluid communication with inflation cannula 860 of catheter assembly 800.

Balloon 830 may be formed from balloon wall or membrane 835 which is selectively inflatable to dilate from a collapsed configuration to a desired and controlled expanded configuration. Balloon 830 may be selectively dilated (inflated) by supplying a fluid into inflation cannula 860 at a predetermined rate of pressure through inflation port 865 (located at proximal end 820). Balloon wall 835 is selectively deflatable, after inflation, to return to the collapsed configuration or a deflated profile. Balloon 830 may be dilated (inflated) by the introduction of a liquid into inflation cannula 860. Liquids containing treatment and/or diagnostic agents may also be used to inflate balloon 830. In one embodiment, balloon 830 may be made of a material that is permeable to such treatment and/or diagnostic liquids. To inflate balloon 830, the fluid may be supplied into inflation cannula 860 at a predetermined pressure, for example, between about one and 20 atmospheres. The specific pressure depends on various factors, such as the thickness of balloon wall 835, the material from which balloon wall 835 is made, the type of substance employed and the flow-rate that is desired. In some embodiments, a balloon may be necessary to temporarily occlude a blood vessel so that the natural flow of blood does not interrupt the procedure by preventing proper placement of the injection needle. In other embodiments, a balloon may be necessary to localize the injection needle near the target region, or ischemic tissue.

Catheter assembly 800 also includes at least two substance delivery assemblies 805a and 805b (not shown; see FIGS. 8B-8C) for injecting a substance into a myocardial infarct region or other treatment region. In one embodiment, substance delivery assembly 805a includes needle 815a movably disposed within hollow delivery lumen 825a. Delivery assembly 805b includes needle 815b movably disposed within hollow delivery lumen 825b (not shown; see FIGS. 8B-8C). Delivery lumen 825a and delivery lumen 825b each extend between distal portion 810 and proximal portion 820. Delivery lumen 825a and delivery lumen 825b may be made from any suitable material, such as polymers and copolymers of polyamides, polyolefins, polyurethanes and the like. Access to the proximal end of delivery lumen 825a or delivery lumen 825b for insertion of needle 815a or 815b, respectively is provided through hub 835 (located at proximal end 820). Delivery lumens 825a and 825b may be used to deliver core-shell particles to a post-myocardial infarct region. In some embodiments, it may be necessary to maximize therapeutic treatment by using a dual needle catheter. Also, a dual needle catheter may be appropriate when two different therapies are desired. For example, the core-shell particles may be dispersed within either a one component or a two component gel system. When the one or two components are injected into the ischemic tissue, the compound may gel, forming a bioscaffolding with the core-shell particles distributed therein. It should be appreciated, however, that a single needle catheter may be appropriate for delivery of the core shell particles. Examples of gel systems may be, but are not limited to, the biomacromolecule systems listed previously.

FIG. 8B shows a cross-section of catheter assembly 800 through line A-A′ of FIG. 8A (at distal portion 810). FIG. 8C shows a cross-section of catheter assembly 800 through line B-B′ of FIG. 8A. In some embodiments, delivery assemblies 805a and 805b are adjacent to each other. The proximity of delivery assemblies 805a and 805b allows each component of the two-component gelation system to rapidly gel when delivered to a treatment site, such as a post-myocardial infarct region.

FIG. 9 illustrates an embodiment of a syringe which may be used pursuant to embodiments of the invention. Syringe 900 may include a body 905, a needle 910 and a plunger 915. A shaft of plunger 915 has an exterior diameter slightly less than an interior diameter of body 905 so that plunger 915 may, in one position, retain a substance in body 905 and, in another position, push a substance through needle 910. Syringes are known by those skilled in the art. In some applications, syringe 900 may be applied directly to a treatment site during an open-chest surgery procedure to deliver core-shell particles to a treatment site.

From the foregoing detailed description, it will be evident that there are a number of changes, adaptations and modifications of the present invention which come within the province of those skilled in the part. The scope of the invention includes any combination of the elements from the different species and embodiments disclosed herein, as well as subassemblies, assemblies and methods thereof. However, it is intended that all such variations not departing from the spirit of the invention be considered as within the scope thereof.

What is claimed is:

1. A composition comprising:
a plurality of particles having a core-shell configuration, each particle comprising:
at least one first biodegradable polymer comprising the shell of the particle;
at least one second biodegradable polymer comprising the core of the particle; and
at least one treatment agent associated with one of the first polymer, the second polymer, or a combination thereof.

2. The composition of claim 1, further comprising, a third biodegradable polymer comprising a second shell wherein the second shell encapsulates at least one core-shell particle.

3. The composition of claim 2 wherein the first polymer, the second polymer, and the third polymer have different degradation rates relative to one another.

4. The composition of claim 2 wherein the first polymer is hydrophobic and the second polymer is hydrophilic.

5. The composition of claim 4 wherein the third polymer is hydrophilic.

6. The composition of claim 2 wherein the first polymer is hydrophilic and the second polymer is hydrophobic.

7. The composition of claim 4 wherein the third polymer is hydrophobic.

8. The composition of claim 2 wherein the first polymer and the third polymer are selected from the group consisting of poly(lactide-co-glycolide), poly(ε-caprolactone), poly(D,L)lactide, and poly(L-lactide) and the second polymer is selected from the group consisting of poly(ethylene glycol), poly(vinyl alcohol), and Pluronics.

9. The composition of claim 2 wherein the second polymer is selected from the group consisting of poly(lactide-co-glycolide), poly(ε-caprolactone), poly(D,L)lactide, and poly(L-lactide) and the first polymer and the third polymer is selected from the group consisting of poly(ethylene glycol), poly(vinyl alcohol), and Pluronics.

10. The composition of claim 2, further comprising at least one treatment agent associated with the third polymer.

11. The composition of claim 2 or 10 wherein the treatment agent is a growth factor selected from the group consisting of vascular endothelial growth factor, basic fibroblast growth factor, acidic fibroblast growth factor, platelet-derived growth factor, platelet-derived endothelial growth factor, placental derived growth factor, insulin-like growth factor 1, insulin like growth factor 2, angiopoietin-1, angiopoietin-2, transforming growth factor-alpha, transforming growth factor-beta, hepatocyte growth factor, stem cell factor, hematopoietic growth factor, granulocyte colony-stimulating factor, granulocyte macrophage colony-stimulating factor, nerve growth factor, growth differentiation factor-9, epidermal growth factor, stromal derived growth factor-1α neurotrophin, erythropoietin, thrombopoieten, myostatin, leukemia inhibitory factor, tumor necrosis factor-alpha, and sonic hedgehog protein.

12. The composition of claim 2 or 10 wherein the treatment agent is a therapeutic agent selected from the group consisting of an anti-proliferative, an anti-inflammatory or immune modulating agent, an anti-migratory, an anti-thrombotic or other pro-healing agent, or a combination thereof.

13. The composition of claim 2 wherein the core, the shell and the second shell are simultaneously produced by an electrospray method.

14. The composition of claim 2 wherein the plurality of particles are in a range from between nanometers to micrometers.

15. The composition of claim 14 wherein the plurality of particles is in a solvent system suitable for injection into a mammal.

16. The composition of claim 2 wherein a surface morphology of the plurality of particles is a function of a ratio of the first biodegradable polymer to the second biodegradable polymer.

17. The composition of claim 16 wherein the surface morphology is smooth.

18. The composition of claim 16 wherein the surface morphology is porous.

19. A system comprising:
a device for forming a plurality of core-shell particles, comprising:
a first cylindrical member;
a second cylindrical member positioned co-axial within the first cylindrical member; and
at least one third cylindrical member positioned in a configuration comprising one of co-axial within the second cylindrical member or adjacent to the second cylindrical member.

20. The system of claim 19, further comprising,
at least one feeder in fluid connection with one of the first, second or third cylindrical members; and
a collection target.

21. The system of claim 20 wherein the collection target is one of (i) a dry collector comprising conductive metal, a non-conductive material with a conductive metal surface, a conductive material with a non-conductive material surface, or an enclosed chamber with circulating or stagnant air or a (ii) a wet collector comprising an aqueous solution or an organic solution.

22. The system of claim 21 wherein the collection target is connected to a power supply and is grounded.

23. The system of claim 21 wherein the collection target is connected to a power supply and is charged.

24. The system of claims 22 or 23 wherein the third cylindrical member is co-axial within the second cylindrical member and the second and third cylindrical members are comprised of a conductive material.

25. The system of claim 24 wherein the first cylindrical member is comprised of an insulating material.

26. The system of claim 25 wherein at least one of the second and third cylindrical members is connected to a power supply capable of applying a charge to the second and third cylindrical members.

27. The system of claim 22 and 23 wherein the third cylindrical member is adjacent to the second cylindrical member and the second and third cylindrical members are comprised of a conductive material.

28. The system of claim 27 wherein the first cylindrical member is comprised of an insulating material.

29. The system of claim 28 wherein at least one of the second and third cylindrical members is connected to a power supply capable of applying a charge to the second and third cylindrical members.

30. The system of claim 27 further comprising a plurality of cylindrical members positioned adjacent to the second cylindrical member, the plurality of cylindrical members comprised of a conductive material.

31. A method of manufacture, comprising:
providing a first solution into a first cylindrical member configured to receive and expel the first solution, the first solution comprising a biodegradable, biocompatible hydrophobic material;
providing a second solution into a second cylindrical member configured to receive and expel the second solution wherein the second cylindrical member is co-axially situated within the first cylindrical member, the second solution comprising a biodegradable, biocompatible hydrophilic material;
providing a third solution into a third cylindrical member configured to receive and expel the third solution wherein the third cylindrical member is situated in a configuration comprising one of co-axially within the second cylindrical member or adjacent to the second cylindrical member, the third solution comprising a biodegradable, biocompatible hydrophilic material;
applying a charge to at least one of the second solution, the third solution, the second cylindrical member or the third cylindrical member;
applying a force to the first solution, second solution and third solution to force the solutions through the cylindrical members; and
collecting resultant particles expelled from the cylindrical members on a collection target wherein the collection target is grounded.

32. The method of claim 31 wherein a tip of the second cylindrical member intersects a plane of an opening of the first cylindrical member and a tip of the third cylindrical member intersects a plane of an opening of the second cylindrical member, the third cylindrical member situated co-axially within the second cylindrical member.

33. The method of claim 31 wherein the third cylindrical member is situated adjacent to the second cylindrical member and a tip of the second cylindrical member and a tip of the third cylindrical member intersect a plane of an opening of the first cylindrical member, the tips of the second and third cylindrical members aligned with one another.

34. The method of claim 33, further comprising, providing a plurality of solutions to a plurality of cylindrical members situated adjacent to the second and third cylindrical members, wherein tips of the plurality of cylindrical members are aligned with the tips of the second and third cylindrical members.

35. The method of claim 31 wherein the material is a polymer or biomacromolecule.

36. The method of claim 31 wherein the hydrophobic material is selected from the group consisting of poly(lactide-co-glycolide), poly(ε-caprolactone), poly(D,L)lactide, and poly(L-lactide).

37. The method of claim 31 wherein the hydrophilic material is selected from the group consisting of poly(lactide-co-glycolide), poly(ethylene glycol), poly(vinyl alcohol), polyvinylpyrrolidone, and Pluronics.

38. The method of claim 31, further comprising, providing at least one treatment agent within one of the first, second and third solutions.

39. The method of claim 38 wherein the treatment agent is a growth factor selected from the group consisting of vascular endothelial growth factor, basic fibroblast growth factor, acidic fibroblast growth factor, platelet-derived growth factor, platelet-derived endothelial growth factor, placental derived growth factor, insulin-like growth factor 1, insulin like growth factor 2, angiopoietin-1, angiopoietin-2, transforming growth factor-alpha, transforming growth factor-beta, hepatocyte growth factor, stem cell factor, hematopoietic growth factor, granulocyte colony-stimulating factor, granulocyte macrophage colony-stimulating factor, nerve growth factor, growth differentiation factor-9, epidermal growth factor, stromal derived growth factor-1α neurotrophin, erythropoietin, thrombopoieten, myostatin, leukemia inhibitory factor, tumor necrosis factor-alpha, and sonic hedgehog protein.

40. The method of claim 28, further comprising, controlling a surface morphology of the resultant particles wherein the surface morphology is controlled as a function of a ratio of hydrophilic material to hydrophobic material.

41. The method of claim 40 wherein the resultant particles have a morphology of one of irregular, spherical or disc-shaped.

42. The method of claim 28, further comprising, controlling a surface morphology of the resultant particles by controlling an operating variable selected from the group consisting of solvent selection, a spray distance, and a collection method wherein the surface morphology is one of irregular, spherical, or disc-like.

43. A system comprising:
a plurality of components, comprising:
a first chamber having a second chamber situated co-axially therein;
a first receiving device in fluid communication with a proximal end of the chamber;
a second receiving device in fluid communication with the second chamber;
a co-axial exit device in fluid communication with a distal end of the first chamber;
means for forcing a substance received from the first receiving device through the chamber to exit the exit device;
means for forcing a substance received from the second receiving device through the chamber to exit the exit device; and
a power supply connected to at least one component.

44. The system of claim 43, further comprising, means for heating the substances.

45. The system of claim 44 wherein the first and second receiving devices are one of a hopper or multi-hopper or spooling or multi-spooling device.

46. The system of claim 44 wherein the exit device is a nozzle, multi-array nozzle, die or multi-array die.

47. The system of claim 44 wherein the collection target is one of (i) a dry collector comprising conductive metal, a non-conductive material with a conductive metal surface, or an enclosed chamber with circulating air or a (ii) a wet collector comprising an aqueous solution or an organic solution.

48. The system of claim 46 wherein the exit device is connected to the power supply, the power supply capable of supplying a charge to the exit device.

49. The system of claim 48 wherein the collection target is connected to the power supply the power supply capable of supplying a charge to the collection target.

50. The system of claim 49 wherein the exit device is charged and the collection target is grounded.

51. The system of claim 49 wherein the exit device is charged and the collection target is oppositely charged to that of the exit device.
